(19) United States
(12) Patent Application Publication
(10) Pub. No.: US 2019/0112246 A1
(43) Pub. Date: Apr. 18, 2019

(54) INTEGRATED TECHNIQUES FOR PRODUCING BIO-METHANOL

(71) Applicant: Ultra Clean Ecolene Inc., Kincardine (CA)

(72) Inventor: Norman J. MacGregor, Kincardine, Ontario (CA)

(21) Appl. No.: 16/091,909

(22) PCT Filed: Feb. 15, 2017
(86) PCT No.: PCT/CA2017/050192

§ 371 Date: Oct. 5, 2018

Related U.S. Application Data
(60) Provisional application No. 62/332,743, filed on May 6, 2016.

(51) Int. Cl. C07C 29/151; C07C 31/04; C02F 3/28; C25B 1/04

(52) U.S. Cl. CPC C07C 29/1518; C25B 1/04; C02F 3/2893; C07C 31/04

(57) ABSTRACT

Methods and systems for producing bio-methanol can include anaerobic digestion of a biomass feedstock to produce biogas including methane and carbon dioxide, partial oxidation of the biogas with oxygen from water electrolysis to produce syngas, synthesizing bio-methanol from the syngas and hydrogen from the water electrolysis, storing the bio-methanol during off-peak electricity demand, intermittently generating electricity from the bio-methanol during peak electricity demand and using such electricity for the water electrolysis. The techniques provide a route for the production of bio-methanol without the engagement of fossil fuels as feedstocks and mitigating fossil fuel derived greenhouse gas emissions from processing and utilization of transportation fuels and commercial or industrial alcohols.

### TECHNICAL FIELD

The technical field generally relates to the production of methanol, and particularly to integrated processes and systems for producing methanol based biofuel from naturally occurring elements.

### BACKGROUND

Liquid biofuel can be produced from a variety of feedstocks and using various different processing technologies. Energy and reactant requirements for conventional liquid biofuel production techniques can lead to technical and economic challenges as well as elevated fossil fuel emissions.

### SUMMARY

The techniques described herein relate to a route for the production of a liquid biofuel without the engagement of fossil fuels as feedstocks or fossil fuel sourced emissions, and more particularly to integrated processes and systems for producing a liquid hydrocarbon-based sustainable bio-methanol. The techniques enable mitigating fossil fuel derived greenhouse gas emissions from processing and utilization of transportation fuels and commercial or industrial alcohols.

In some implementations, a method is provided for producing bio-methanol, comprising:
supplying biomass to an anaerobic digester for producing biogas comprising methane and carbon dioxide;
supplying the biogas and oxygen sourced from water using renewable and/or nuclear-sourced electricity to a partial oxidation unit to produce non fossil fuel-sourced syngas;
supplying the syngas with hydrogen sourced from water using renewable and/or nuclear-sourced electricity to a synthesis unit for producing bio-methanol;
during electricity demand below a base threshold:
   supplying at least a portion of the bio-methanol to storage (e.g., compressed storage) for use as transportation fuel and/or other applications (e.g., commercial/industrial alcohol); and
during electricity demand over a base threshold:
   supplying at least a portion of the bio-methanol to a generator for intermittently producing electricity (e.g., during peak demand);
supplying water to a water electrolysis unit to produce electrolysis oxygen and electrolysis hydrogen;
supplying at least a portion of the electrolysis hydrogen as at least part of the hydrogen used in the synthesis unit; and
supplying at least a portion of the electrolysis oxygen as at least part of the oxygen used in the partial oxidation unit.

In some implementations, the biomass comprises manure, municipal waste, agricultural waste, organic waste, sewerage, purpose grown biomass, and/or cellulose.

In some implementations, the anaerobic digester further produces sulphur and/or fertilizer, and optionally requires supplement heat energy for maximum biogas production.

In some implementations, the process includes heating the anaerobic digester using by-product heat generated by the partial oxidation unit.

In some implementations, the process includes heating the anaerobic digester using by-product heat generated by the water electrolysis unit.

In some implementations, the oxygen supplied to the partial oxidation unit consists of the electrolysis oxygen.

In some implementations, the oxygen supplied to the partial oxidation unit is obtained from an oxygen storage vessel.

In some implementations, the syngas supplied to the synthesis unit consists of the syngas produced by the partial oxidation unit.

In some implementations, the hydrogen supplied to the synthesis unit consists of the electrolysis hydrogen.

In some implementations, the transportation bio-methanol is used as fuel for automobile engines, diesel engines, fuel cells and/or base energy platform for refinery upgrading to aircraft fuel.

In some implementations, the water electrolysis unit further produces deuterium.

In some implementations, at least a portion of the deuterium is supplied to a nuclear reactor facility.

In some implementations, the process also includes the following:

during electricity demand over an upper value:
powering the water electrolysis unit using electricity obtained from a generator fuelled with a portion of the stored bio-methanol;

during electricity demand below a lower value:
powering the water electrolysis unit, and optionally hydrogen and oxygen compressors, using electricity obtained from a source supplied by renewable and/or nuclear energy sources and/or from independent renewable electricity generators.

In some implementations, the base threshold is relatively constant and pre-determined. In some implementations, the upper and lower values are the same. In some implementations, the upper and lower values and the base threshold are the same.

In some implementations, the process includes regulating the base threshold over time to maintain the overall greenhouse gas neutrality of the process.

In some implementations, the process includes controlling electricity input into the water electrolysis unit and controlling the electricity generation from the bio-methanol to maintain the overall greenhouse gas neutrality of the process, and reducing negative impacts of electricity supply/demand characteristics.

In some implementations, a system is provided for producing bio-methanol, comprising:
an anaerobic digester unit for producing biogas comprising methane and carbon dioxide;
a partial oxidation unit for receiving the biogas and configured to produce syngas;
a synthesis unit for receiving the syngas and carbon neutral hydrogen, and configured to produce bio-methanol;
a power control assembly configured to
   supply at least a portion of the bio-methanol to a generator for producing electricity, during critical electricity demand over an upper threshold; and
   supply at least a portion of the bio-methanol to storage for use as transportation fuel, during electricity demand below a lower threshold;
a carbon neutral water electrolysis unit to produce carbon neutral oxygen and hydrogen;
a hydrogen supply and storage assembly configured to supply at least a portion of the electrolysis hydrogen as at least part of the hydrogen used in the synthesis unit; and
an oxygen supply and storage assembly configured to supply at least a portion of the electrolysis oxygen as at least part of the oxygen used in the partial oxidation unit.

In some implementations, a method is provided for producing bio-methanol, comprising:
supplying biomass to an anaerobic digester for producing biogas comprising methane and carbon dioxide;
supplying the biogas and oxygen to a partial oxidation unit to produce syngas;
supplying the syngas and hydrogen to a synthesis unit for producing bio-methanol;
supplying water (e.g., distilled water) to a water electrolysis unit to produce electrolysis oxygen and electrolysis hydrogen;
supplying at least a portion of the electrolysis hydrogen as at least part of the hydrogen used in the synthesis unit;
supplying at least a portion of the electrolysis oxygen as at least part of the oxygen used in the partial oxidation unit; and
controlling electrical input provided to the water electrolysis unit, comprising:
   during electricity demand over an upper value:
      powering the water electrolysis unit using electricity obtained from renewable energy sources and/or nuclear energy sources; and
   during electricity demand below a lower value:
      powering the water electrolysis unit using electricity obtained from the selected supply.

In some implementations, a method is provided for producing bio-methanol, comprising:
supplying biomass to an anaerobic digester for producing biogas comprising methane and carbon dioxide;
optionally, supplying one or more by-products generated by the anaerobic digester (e.g., sulfur, fertilizer), for example when derived from organic waste, sanitary sewerage and/or manures, to corresponding storage units and/or to further processing for sale or utilization;
supplying biogas and oxygen to a partial oxidation unit to produce syngas (e.g., non fossil fuel sourced sungas);
supplying syngas and hydrogen to a synthesis unit for producing bio-methanol;
supplying water (e.g., distilled water) to a water electrolysis unit to produce electrolysis oxygen and electrolysis hydrogen;
supplying at least a portion of the electrolysis hydrogen as at least part of the hydrogen used in the synthesis unit;
supplying at least a portion of the electrolysis oxygen as at least part of the oxygen used in the partial oxidation unit.
integrating bio-methanol storage, electricity generation and water electrolysis, comprising:
   storing an inventory of bio-methanol;
   controlling electricity input into the water electrolysis unit, comprising:
      monitoring electricity demand;
      based on the monitored electricity demand, periodically:
         combusting a portion of the bio-methanol retrieved from the inventory to provide bio-methanol- and/or biogas-generated electricity; and
         utilizing the bio-methanol-generated electricity in the water electrolysis unit.

In some implementations, a process is provided for integrating a water electrolysis unit and bio-methanol storage facility:

monitoring electricity demand;

during electricity peak demand:
diverting bio-methanol from storage to electricity generation to produce biofuel-generated electricity;
reducing or ceasing system electricity utilization for operating the water electrolysis unit; and
utilizing the bio-methanol- and/or biogas-generated electricity for operating the water electrolysis unit; and

during electrical system demand below the peak:
storing bio-methanol for distribution for use as a transportation biofuel (e.g., as a greenhouse gas neutral fuel);
ceasing generation of the bio-methanol generated electricity; and
increasing use of the system electricity for the water electrolysis unit.

In some implementations, there is provided a method for producing bio-methanol, comprising: supplying a feedstock that comprises or consists of biomass to an anaerobic digester for producing biogas comprising methane and carbon dioxide; supplying all or some of the biogas, directly or indirectly, to a partial oxidation unit to produce non fossil fuel-sourced syngas, wherein oxygen is also supplied thereto; supplying the syngas, directly or indirectly, to a synthesis unit for producing bio-methanol, wherein with hydrogen is also supplied thereto; supplying water to a water electrolysis unit to produce electrolysis oxygen and electrolysis hydrogen; supplying at least a portion of the electrolysis hydrogen as at least part of the hydrogen used in the synthesis unit; and supplying at least a portion of the electrolysis oxygen as at least part of the oxygen used in the partial oxidation unit.

There is also provided a method for operating a bio-methanol production plant without fossil fuels, comprising: supplying a feedstock consisting of biomass to an anaerobic digester for producing biogas comprising methane and carbon dioxide; supplying a feed consisting of the biogas and oxygen sourced from water using renewable and/or nuclear-sourced electricity to a partial oxidation unit to produce syngas; supplying a feed consisting of the syngas and hydrogen sourced from water using renewable and/or nuclear-sourced electricity to a synthesis unit for producing bio-methanol; during off-peak electricity demand, supplying at least a portion of the bio-methanol to storage; and during peak electricity demand, supplying at least a portion of the bio-methanol to a generator for intermittently producing bio-methanol generated electricity; electrolyzing water in a water electrolysis unit to produce electrolysis oxygen and electrolysis hydrogen, and during peak electricity demand using the bio-methanol generated electricity in the water electrolysis unit; supplying at least a portion of the electrolysis hydrogen as at least part of the hydrogen used in the synthesis unit; and supplying at least a portion of the electrolysis oxygen as at least part of the oxygen used in the partial oxidation unit.

There may also be a system for producing bio-methanol, comprising an anaerobic digester unit for producing biogas comprising methane and carbon dioxide; a partial oxidation unit for receiving the biogas and configured to produce syngas; a synthesis unit for receiving the syngas and carbon neutral hydrogen, and configured to produce bio-methanol; an assembly configured to supply at least a portion of the bio-methanol to a generator for producing electricity, during critical electricity demand over an upper threshold, and supply at least a portion of the bio-methanol to storage for use as transportation fuel or as a commercial or industrial alcohol, during electricity demand below a lower threshold; a water electrolysis unit to produce oxygen and hydrogen; a hydrogen supply and storage assembly configured to supply at least a portion of the electrolysis hydrogen as at least part of the hydrogen used in the synthesis unit; and an oxygen supply and storage assembly configured to supply at least a portion of the electrolysis oxygen as at least part of the oxygen used in the partial oxidation unit.

The system can include one or more features as recited above or herein in terms of elements of each unit, each stream (input and output streams of each unit), or the interconnection or operation of the units.

In addition, there may also be a generator assembly for integration into a bio-methanol production facility, the assembly including a liquid inlet for periodically receiving bio-methanol; a generator unit for combusting the periodically received bio-methanol in order to combust the same and generate electricity; a electricity output line for transmitting the electricity generated from combustion to the bio-methanol production facility (e.g., to at least a water electrolysis unit); and a control unit for controlling operation such that, during peak electricity periods, the generator receives and combusts bio-methanol for electricity generation and the electricity output line supplies electricity to the bio-methanol production facility, and during low electricity periods the generator ceases combustion and supply of electricity to the bio-methanol production facility. The control unit can include modules for receiving information regarding electricity demand and price levels, and modules for receiving information regarding bio-methanol storage levels (e.g., from instrumentation such as tank level detectors). The control unit can also be coupled to valves that control the supply of bio-methanol to the generator, and to the generator to control certain operating parameters of the combustion and electricity generation in order to produce a predetermined rate of electricity that may be coordinated with the electricity requirements of the unit(s) of the bio-methanol production facility (e.g., the water electrolysis unit). The control unit can also be coupled to the water electrolysis unit or another unit to which electricity is supplied, in order to control the generator to supply the appropriate electricity. The control unit can also be coupled to hydrogen and oxygen storage units, which store the products of the water electrolysis unit, to ensure that hydrogen and oxygen levels are suitably maintained for operation of the bio-methanol production facility. The generator assembly can be integrated into an existing bio-methanol production facility as part of retrofitting or can be part of a newly designed and built facility.

In some implementations, the system, method and/or process includes additional features and/or steps as recited further above or herein. For example, the process may include the features that, during electricity demand below a base threshold, at least a portion of the bio-methanol is supplied to storage for use as transportation fuel and/or commercial/industrial alcohol; and/or during electricity demand over a base threshold, at least a portion of the bio-methanol is supplied to a generator for intermittently producing electricity. The production and/or the inventory or the bio-methanol can be controlled according to the proportion of the bio-methanol supplied for distribution or use as transportation fuel and/or commercial/industrial alcohol as well as for generation of electricity, which may be used in the process (e.g., in the water electrolysis unit).

### BRIEF DESCRIPTION OF THE DRAWINGS

FIG. 1 is a block diagram of an integrated bio-methanol production process with greenhouse gas neutrality.

FIG. 2 is a block diagram of a biomass anaerobic digester.

FIG. 3 is a block diagram of a water electrolysis unit operation.

FIG. 4 is a block diagram of a partial oxidation unit.

FIG. 5 is a block diagram of a synthesis unit and tank farm.

FIG. 6 is a block diagram of a generator.

FIG. 7 is a block diagram of several integrated units and illustrating the electricity source in terms of its supply-demand balance characteristics.

FIG. 8 is another block diagram of an integrated bio-methanol production process.

FIG. 9 is a block diagram of part of a bio-methanol production process.

FIG. 10 is another block diagram of part of a bio-methanol production process.

FIG. 11 is another block diagram of part of a bio-methanol production process.

FIG. 12 is another block diagram of part of a bio-methanol production process.

FIG. 13 is a graph of throughput/production versus electricity source for an example bio-methanol production process.

### DETAILED DESCRIPTION

Various techniques are described herein for bio-methanol production. In some implementations, systems and processes are provided for the production of bio-methanol (which may be referred to here as ECOLENE®). The bio-methanol can be dedicated as a liquid transportation biofuel, as a commercial/industrial alcohol, and/or as a liquid biofuel for generating greenhouse gas neutral electricity particularly during peak electrical demand periods. The bio-methanol can also be dedicated as a liquid storage medium for surplus and low-demand nuclear and/or renewable electricity as well as a novel medium for temporary storage of captured greenhouse gases from decomposed biomass for delayed release back to the atmosphere for balancing via photosynthesis.

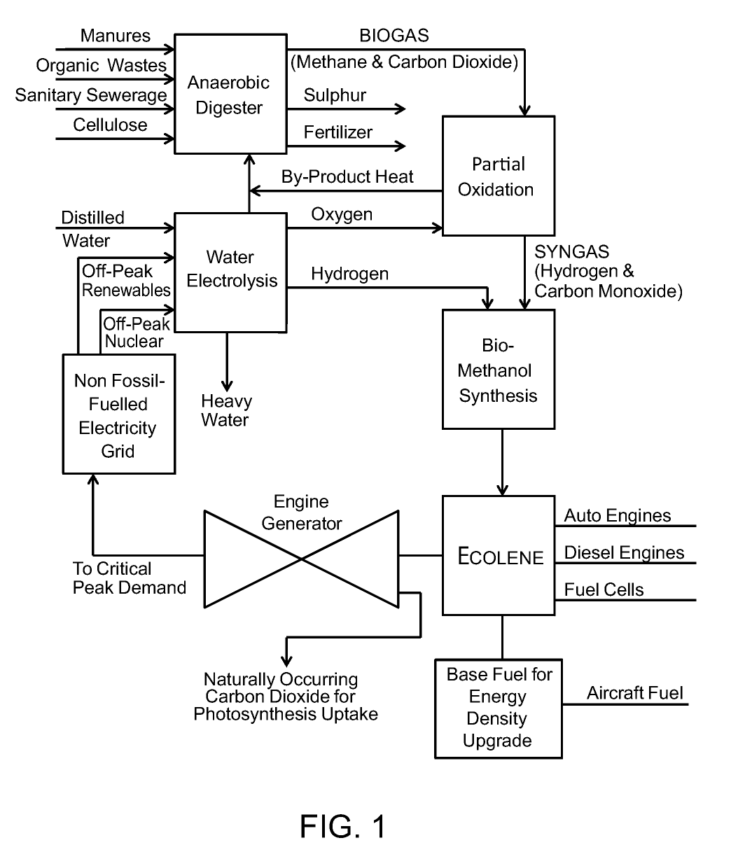

Referring to FIG. 1, the system can include integrated units for bio-methanol production and can include an anaerobic digester unit, a partial oxidation unit, a synthesis unit, a storage facility, a water electrolysis unit, and a modulating electricity generator.

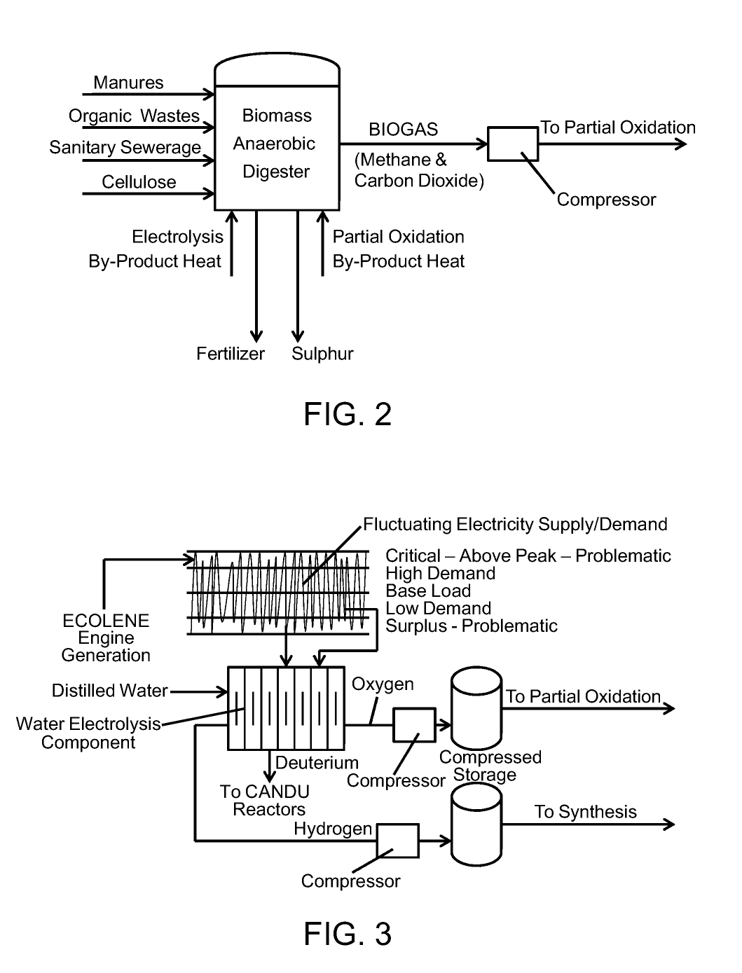

Referring to FIGS. 1 and 2, in some implementations the anaerobic digester is configured to receive one or more biomass feedstocks, such as manures, organic wastes, sanitary sewerage, cellulose (e.g., pulverized cellulose), and so on. The biomass feedstocks can be sourced locally and can include a combination of different hydrocarbon and carbohydrate sources. The digester can be operated to produce biogas as well as sulphur and fertilizer by-product streams. The sulphur can be harvested incrementally and the composted fertilizer can also be recovered periodically, as by-products. The fertilizer can be recovered as a coliform-free material and can be processed for sale and/or used in a dedicated biomass production facility (e.g., a greenhouse) that may also use CO₂ that is produced by the process. Both the fertilizer and the CO₂ generated by the process can be stored and then supplied as needed to a biomass production facility (e.g., during certain biomass production cycles). In some cases, the biomass that is produced can then be harvested as part of the feedstock supplied to the anaerobic digester. A biogas storage unit can be provided to receive and store biogas from the digester. A biogas compressor can be provided to operate the digester at or near steady state in order to prevent exhausting and/or flaring of biogas during surplus biogas production periods and other times of the processing. The biogas storage can be monitored and controlled to retrieve and supply controlled amounts of the biogas to the partial oxidation unit, for example. Such control can also incorporate input from other process units. The biogas production can be monitored and controlled to obtain a composition within a pre-determined range, particularly with respect to the stoichiometric balance of methane and carbon dioxide, for example to maximize production and utilization.

In some implementations, biogas can be burned directly in the generator, for example in periods of biogas overproduction and/or during outages of partial oxidation and/or synthesis reactors to avoid emissions. The generator unit can include combustion generator devices that are adapted to receive biogas and/or bio-methanol streams as fuel (alternately and/or simultaneously), and/or the generator unit can include multiple generator devices each dedicated to a given fuel (e.g., a biogas-receiving generator, a bio-methanol-receiving generator, etc.).

Referring to FIGS. 1 and 3, in some implementations the water electrolysis unit is configured to receive distilled water and electricity from non-fossil fuel sources. The water can be obtained from a water distillation unit or another type of water purification unit that may be located on site or proximate to the water electrolysis unit, for example. Energy required for water distillation can be obtained in whole or in part from renewable sources, such as biomass or bio-methanol combustion. The water electrolysis unit can be fully variable, fully interruptible and outfitted with compressors and storage vessels to ensure a constant regulated supply of output (oxygen and hydrogen) are available during interruption and/or high electricity demand periods. By-product heat from the water electrolysis unit can be captured and delivered to the digester and/or to pre-treatment units for pre-treating the biomass prior to entering the digester. The by-product heat recovery can facilitate temperature control of the digester for optimizing microbial production when appropriate. The by-product heat can be supplied to cooling fans or towers when the heat is not required elsewhere in the process. In addition, the water electrolysis unit can include deuterium harvesting capability, for recovering deuterium (heavy water) for use as a heat transfer medium and/or in medical applications. The water electrolysis unit can thus be configured and operated to promote production of deuterium-rich liquid. For example, the water electrolysis unit can include a cascade of electrolysis chambers for concentrating the deuterium in each subsequent chamber until pure deuterium is produced, or there may be a separate deuterium harvester/separator that is coupled to the water electrolysis unit to receive deuterium-enriched liquid that can be further separated into a substantially pure deuterium via chemical exchange and/or distillation methods. The electrolysis-derived heavy water can be used in a nuclear reactor heat transfer system (e.g., part of a CANDU™ facility).

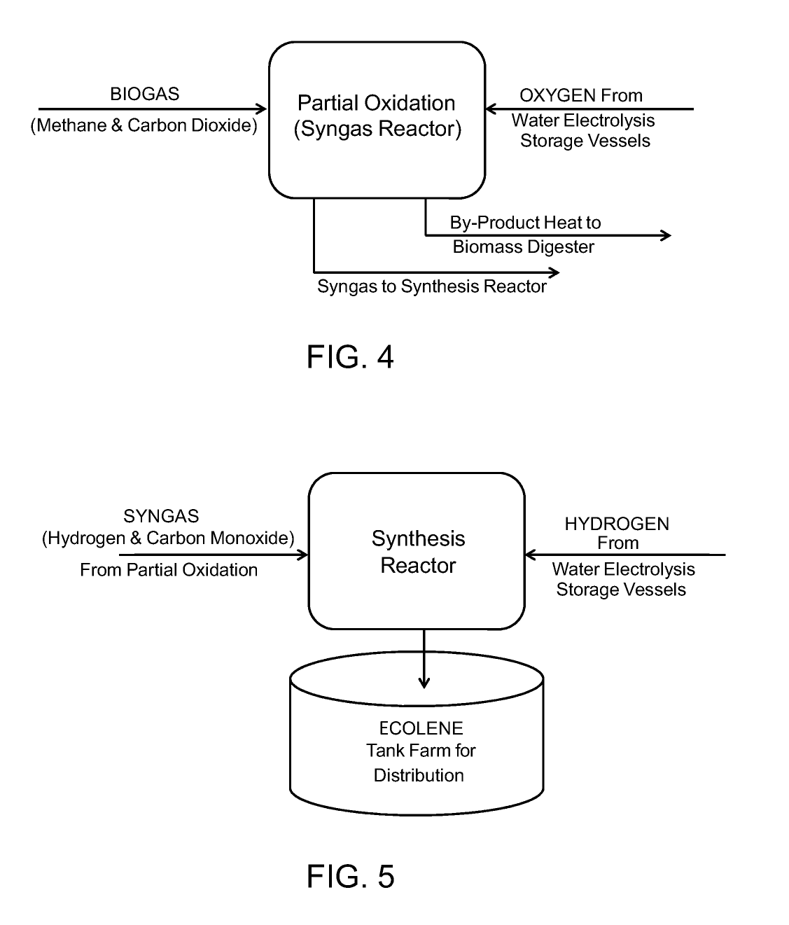

Referring to FIGS. 1 and 4, in some implementations the partial oxidation unit is fluidly connected with the biogas storage facility and/or the digester, to receive biogas to be burned using compressed oxygen sourced from the water electrolysis unit to produce syngas comprising or substantially consisting of hydrogen and carbon monoxide.

Referring to FIGS. 1 and 5, in some implementations the syngas together with compressed hydrogen from water electrolysis are supplied to a synthesis unit configured to produce non fossil fuel-based bio-methanol, which may be referred to herein as ECOLENE®.

Still referring to FIGS. 1, 5 and 6, the bio-methanol can be supplied to a storage facility, e.g., tank farm, which can be monitored and controlled in various ways that will be described herein. The bio-methanol storage facility can be configured for distribution as well as periodic supply to a generator for electricity generation. In some implementations, the bio-methanol storage facility is configured with sufficient tank storage inventory or capacity to enable periodic electricity generation, for example during critical peak demand. The tank storage capacity can therefore be co-ordinated with electrolysis electricity demand and peak non fossil fuelled electricity demand. The storage facility can also include piping, monitoring instrumentation, pumps and control units to manage the storage and the supply of the bio-methanol.

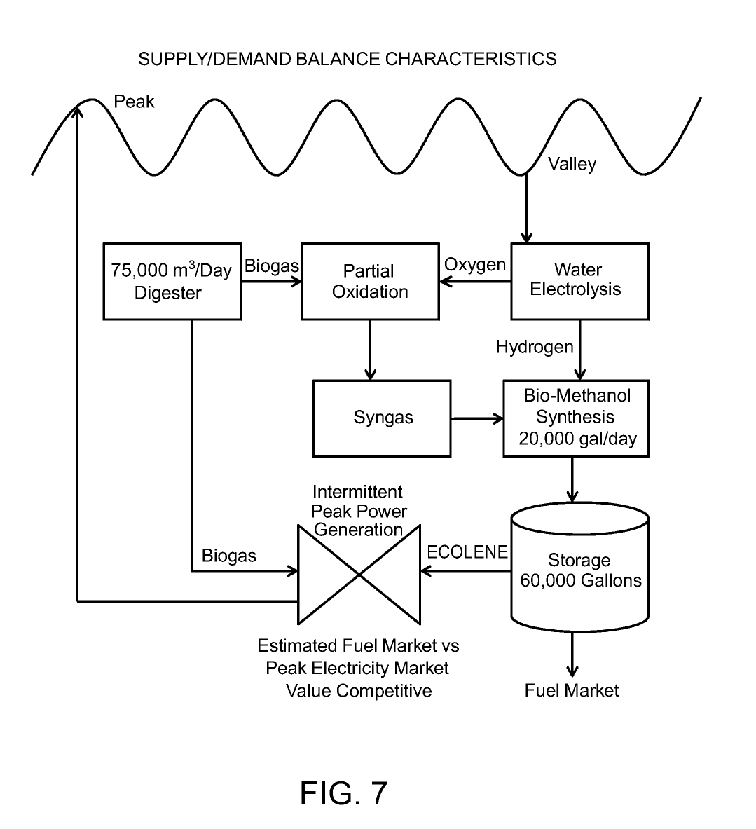

In some implementations, the capacity to intermittently utilize surplus and/or low demand electricity in variable amounts to produce non fossil-sourced hydrocarbons with the capacity to intermittently generate critical and high demand electricity in variable amounts can facilitate the increasing need to balance electricity supply with electricity demand. The capacity to produce bio-methanol during low electricity demand and use the bio-methanol to generate electricity during high electricity demand will help reduce demand charges and improve the quality of electricity. In some scenarios, time-of-day pricing by electricity system operators can be used to determine the value for using surplus electricity capacity for purchasing low demand electricity and a charge for demand. The capacity to generate electricity using bio-methanol ECOLENE® and/or biogas can be determined by the steady state capacity of the biogas using ECOLENE® as a back-up biofuel. For example, a 20,000 US gal/day “regional” bio-methanol plant may use 75,000 m³ biogas/day, which is generally reflected in FIG. 7.

Time-of-use pricing of electricity can vary depending on various factors and locations. For example, in some jurisdictions, off-peak electricity rates can apply from approximately 8:00 PM-7:00 AM and can have a cost that is about 65-75% of the mid-peak rate and about 30-55% of the on-peak rate.

In some implementations, the capacities of the different units can be coordinated with factors based on electricity demand cycles, estimated fuel market, and the like. In some scenarios, the digester is sized and operated to produce between 25,000 m³/day and 200,000 m³/day biogas, or between 50,000 m³/day and 100,000 m³/day biogas; the bio-methanol synthesis unit is sized and operated to produce between 5,000 gal/day and 100,000 gal/day of bio-methanol, or between 15,000 gal/day and 25,000 gal/day; and the bio-methanol storage facility has a capacity of between 15,000 gallons and 100,000 gallons, or between 40,000 gallons and 80,000 gallons of the biofuel. Subject to biomass availability, much larger bio-methanol plants can be implemented in the proximity of large nuclear and/or renewable electricity generating sites.

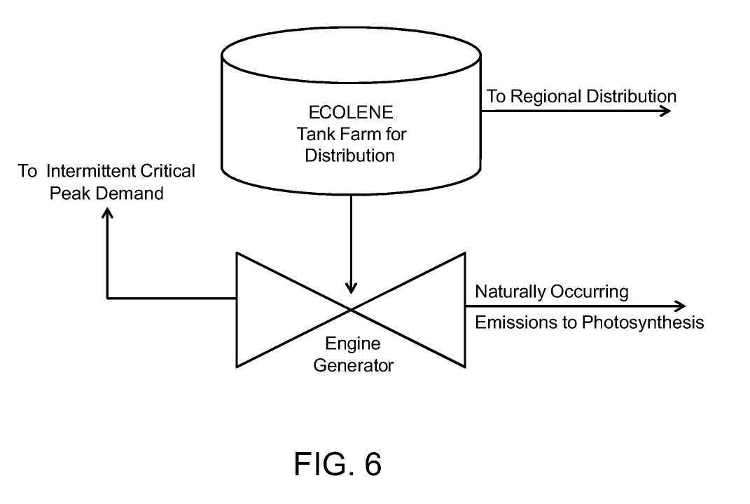

Referring to FIG. 6, a generator can be provided to receive bio-methanol from the storage facility and provide electricity to the water electrolysis unit. The generator may be specially designed and dedicated for the combustion of bio-methanol to produce electricity without emitting fossil fuel sourced greenhouse gases. The generator can be configured to receive different fuels, which may be liquid non fossil-sourced fuels only or a combination of liquid non fossil-sourced fuels including biogas. The combustion of the bio-methanol and/or biogas would be substantially free of fossil sourced greenhouse gas emissions that would be associated with the combustion of fossil fuels, for example. By-product heat from the generator can also be used in the process, e.g., for optimizing the microbial production in the digester.

An integration assembly can be provided to integrate different units of the system. For example, the integration assembly can include the generator, inlet bio-methanol fuel piping, electrical supply lines for supplying bio-methanol generated electricity to the water electrolysis unit, a control unit coupled to the piping and/or valves for controlling the periodic operation of the generator, which may be done according to input variables that include electricity demand levels to determine the timing of peak demand, as well as various detection and monitoring devices such as temperature sensors, pressure sensors and/or flow rate meters and/or actuators. The integration assembly may include an automation apparatus, such as a computer, configured to control the integration automatically in response to the input variables to ensure pressure/temperature and processing duration for the conversion process (e.g., space, gas, velocity).

Various techniques described herein can be used in the context of a carbon capture, carbon storage, carbon trade, carbon credit, and carbon tax systems.

Production of ECOLENE® can enable a liquid hydrocarbon to be commercially synthesized by controlled digestion of waste biomass as feedstock to capture and utilize methane and carbon dioxide to produce a biofuel rather than enter the atmosphere directly as greenhouse gases. By utilizing only renewable- and/or nuclear-sourced electricity, to decompose water to produce the essential elements of hydrogen and oxygen, unlike other methanol synthesis processes which use fossil fuel-sourced input streams, ECOLENE® production enables its emissions of carbon dioxide to remain more in atmospheric balance through photosynthesis.

In some implementations, the system can be a regional hub that is located to serve a remote solar farm, a remote hydraulic generation facility, a remote wind farm and/or an ocean energy facility where conventional grids or related infrastructure are inadequate or do not exist. Bio-methanol can thus be a particularly advantageous source of electricity storage and/or a liquid carrier/transporter of electron energy.

In some implementations, the bio-methanol can also be used as a liquid fuel for various conventional and/or hybrid transportation power trains, as well as other methods. Thus, using biomass, water and variable volumes of renewable and/or nuclear sourced electricity during low electricity system demand, as described herein, can enable bio-methanol to be used to power internal combustion engines for conventional power trains, on-board generators for hybrid and/or all electric power trains, carry hydrogen for fuel cell powered electric drives and/or generate electricity during high electricity demand, qualifying such bio-methanol to be a liquid electricity storage medium “battery”. Bio-methanol production, storage inventory and distribution can be managed to facilitate a plurality of end-uses that can be coordinated with advantageous time periods (e.g., electricity demand cycles), locations (e.g., regional, infrastructure-deficient, etc.), as well as various cost/economic factors.

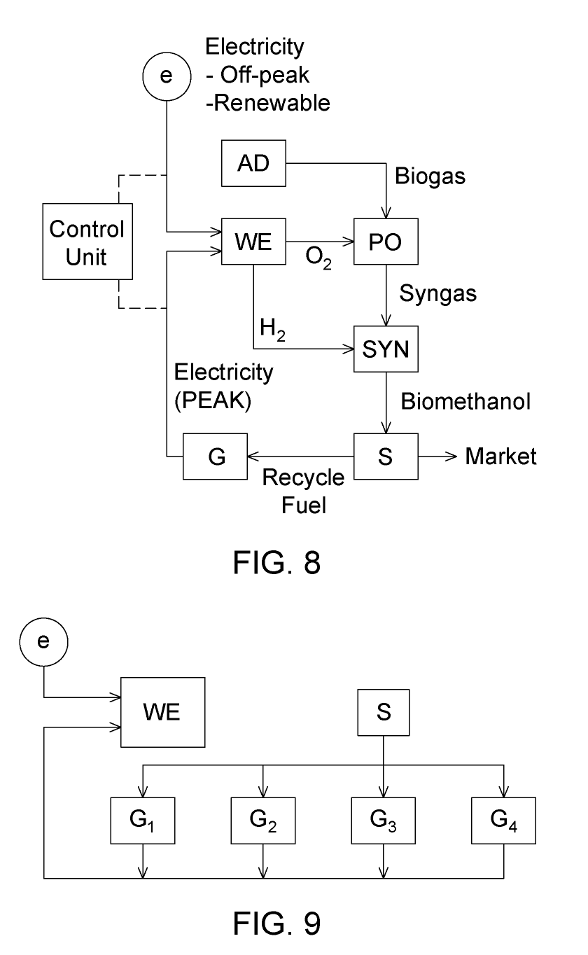

Referring to FIG. 8, the overall bio-methanol fuel production process is illustrated where a control unit is coupled to both the electrical output of the generator (G) and an electrical line from an external electricity source (e), which may include electricity from an electricity grid dominated with renewable sources to ensure the electricity flow is carbon neutral. The control unit can be configured to receive information regarding the bio-methanol production process as well as the external electricity source(s), including cost information for external electricity as well as for inputs (e.g., biomass feedstocks) and outputs (e.g., bio-methanol) of the production system. The control unit can be configured to balance the electricity sources (i.e., internal and external) to minimize cost or to reduce cost while prioritizing more sustainable electricity sources.

Referring to FIG. 9, a water electrolysis unit (WE) can receive electricity from both external sources (e) and internal sources (G₁ to Gₙ). In some scenarios, it may be advantageous to provide multiple generators (G₁ to Gₙ) which can be operated individually or together depending on the electricity demand from the water electrolysis unit (WE). For example, during high throughput/production periods and peak demand, multiple or all of the generators can be operated to produce electricity; while during lower throughput/production periods and/or off-peak, only some or none of the generators can be operated to produce electricity. Multiple smaller generators, all of which can be coupled to a central control unit, can thus be used in a modular fashion to tailor the electricity generation in a flexible manner than can adapt to both external electricity cost and availability and the production mode (e.g., high production, start-up, turndown, upset, etc.) of the bio-methanol production process.

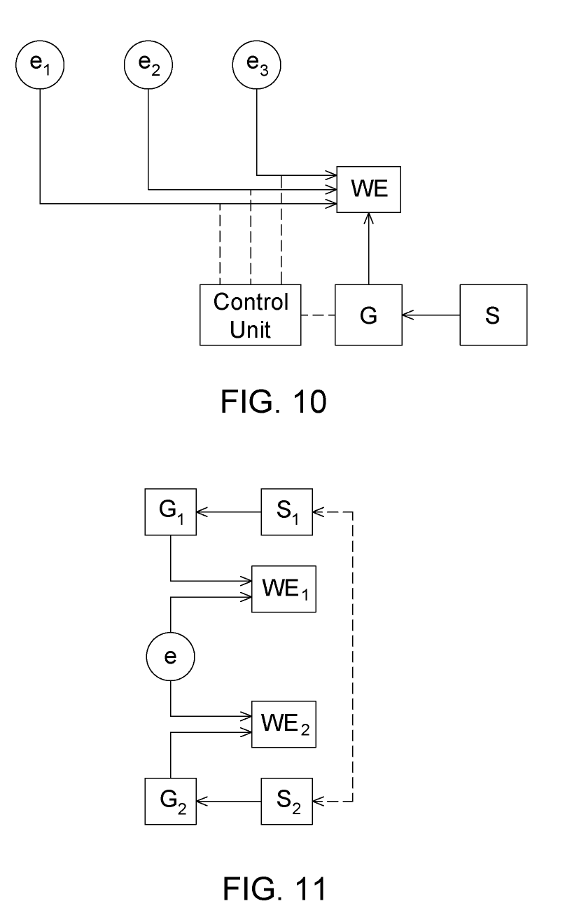

Referring to FIG. 10, the water electrolysis unit (WE) can be coupled to multiple external electricity sources (e₁ to e₃), each of which can originate from a different electricity generation method. For example, a first external electricity source (e₁) may be wind-generated, a second external electricity source (e₂) may be hydro-generated, a third external electricity source (e₃) may be nuclear-generated, while other external electricity sources may come from various other renewable sources, some of which have been mentioned above. By coupling the bio-methanol production process to multiple external electricity sources, access to renewable electricity can be more robust particularly when some of the output from the renewable sources is inconsistent or difficult to predict in terms of availability and/or cost. For example, certain renewable energy sources are weather dependent (e.g., wind) and thus by providing multiple external sources, the process can operate more efficiently. In addition, the control unit can be configured to select and balance the electricity sources that are used for the water electrolysis unit based on fluctuations in each external electricity source.

Referring to FIG. 11, multiple water electrolysis units can be provided and in some cases can employ one or more common external electricity source (e). The multiple water electrolysis units can be part of the same overall bio-methanol production process or they can be part of two distinct and potentially remote processes, e.g., provided in two different regional locations. Each water electrolysis unit (WE₁ and WE₂) can be coupled to its own generator (G₁ and G₂ respectively), which can in turn be coupled to two different storage facilities (S₁ and S₂ respectively) or to a single central storage facility. This general configuration can be particularly advantageous for implementing multiple bio-methanol production plants in a plurality of remote locations that are nevertheless serviced by a common electrical grid and/or by common external electrical sources. In addition, a bank of generators can include a primary generator as well as backup generators, which can come online quickly and periodically to facilitate avoiding spikes in peak demand. Multiple generators can thus be particularly advantageous when there are sudden, large and/or unpredictable spikes in peak demand, by facilitating rapid adjustment.

In some implementations, the primary generator (G₁) can be designed and provided to be able to respond to normal electricity requirements during peak demand periods and typical operation of the bio-methanol production plant, while a secondary or backup generator (G₂) is a smaller unit designed for more occasional operation during sudden peaks, emergency demand periods, and/or when bio-methanol price is lower than external electricity cost. In some implementations, one or more generators can be designed to utilize the bio-methanol as the dedicated fuel, while one or more additional generators are provided for use with other fuel sources (e.g., biogas) or as fuel-neutral units that can receive methanol, biogas and/or other fuel sources for electricity generation.

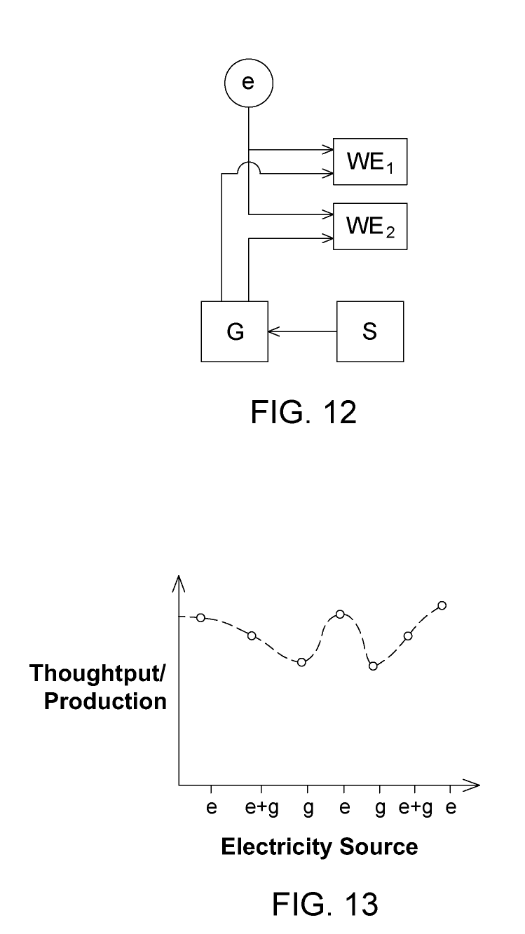

Referring to FIG. 12, the bio-methanol production process can include multiple water electrolysis units (WE₁ and WE₂) that are part of the same production plant and are operated in accordance with electricity sourcing strategy and the bio-methanol production mode. For example, during low throughput/production (e.g. during start-up or turndown modes, maintenance, or feedstock modification) a single water electrolysis unit may be used and it may be supplied with electricity based on the above-described methods by using off-peak electricity from the external source (e) and bio-methanol generated electricity during peak periods. As the production process ramps up, the second water electrolysis unit can come online and can be supplied by both external and internal sources of electricity, as described above. A bank of multiple water electrolysis units can provide additional flexibility for bio-methanol production processes, particularly when the plants have variable throughputs and production.

In addition, the production rate of the process can also be controlled based on electricity availability and cost. For example, during peak demand, the production rate can be decreased in conjunction with using bio-methanol to generate electricity for operating the water electrolysis unit(s). This can be particularly advantageous in the case that the bio-methanol market price is high and/or when the biomass feedstock cost is high, thereby reducing the consumption of bio-methanol for generating electricity while keeping the process operational during peak demand periods. Alternatively, when bio-methanol price and feedstock cost are low, the production rate can be maintained at substantially the same levels as during off-peak operations.

Turning to FIG. 13, an example of modulating throughput and production rate of the process based on the different electricity inputs (e) and/or (G) is illustrated. One can also integrate the cost of biomass feedstocks and the price of the bio-methanol into the control strategy which can be implemented in automated fashion by a control unit that is coupled to the various units of the process.

Advantageously, off-peak external electricity consists of electricity from non-fossil fuel sources. Various examples of non-fossil fuel sources of electricity are provided further above. Further examples are (i) when nuclear reactors are modulated or when primary nuclear sourced steam is being quenched, (ii) when wind energy generation is being strategically curtailed, (iii) when hydro-energy is being spilled as part of a supply management strategy. A number of variable electricity sources can be used.

In addition, since water electrolysis units can incrementally and quickly modulate demand, utilizing water electrolysis units in the context of the techniques described herein facilitates critical load manipulation. Electrolysis interruption is ideally avoided and thus leveraging the bio-methanol for generating electricity dedicated for maintaining electrolysis operation facilitates efficient operation of the process.

In some implementations, the generator (G) is a dedicated bio-methanol combustion unit that is designed and operated for use with 100% methanol as fuel. Alternatively, the generator can be used for various different fuel types, including methanol. In some implementations, the combustion gas generated by the generator(s) is recuperated and reused either within the bio-methanol production process or in other processes. For instance, in some scenarios, the CO₂ in the combustion gas can be separated and reused in the process, in another system (e.g., greenhouses for photosynthesis and production of biomass), and/or in a capture/sequestration system. The CO₂ in the combustion gas can be prepared and supplied directly to a CO₂-utilization facility or can be captured from the combustion gas and stored as substantially pure CO₂ for use. Heat generated by the generator can also be used in a biomass generation facility, such as a greenhouse, or other external or internal units. In some scenarios, at least one of the generators can be portable to facilitate relocation as need be, e.g., between two remote process locations.

Units and components of the systems described herein can also be used and configured in various ways. For example, certain unit operations can be provided as a serial or parallel bank of units. Another example is that processes described herein can be adapted for production of liquid biofuel other than bio-methanol by periodically using liquid biofuel as a source of electricity for one or more units during peak demand periods, particularly when such electricity is supplied to a water electrolysis unit or another unit having similar electricity requirements. In addition, multiple generators can be provided in parallel in order to process different amounts of bio-methanol to produce electricity for the water electrolysis unit depending on the electricity demand, the electrolysis electricity demand and/or the inventory of bio-methanol.

1. A method for producing bio-methanol, comprising:
supplying biomass to an anaerobic digester for producing biogas comprising methane and carbon dioxide;
supplying the biogas and oxygen sourced from water using renewable or nuclear-sourced electricity to a partial oxidation unit to produce non fossil fuel-sourced syngas;
supplying the syngas with hydrogen sourced from water using renewable or nuclear-sourced electricity to a synthesis unit for producing bio-methanol;
during electricity demand below a base threshold:
supplying at least a portion of the bio-methanol to storage; and
during electricity demand over a base threshold:
supplying at least a portion of the bio-methanol to a generator for intermittently producing bio-sourced electricity;
supplying distilled water to a water electrolysis unit to produce electrolysis oxygen and electrolysis hydrogen;
supplying at least a portion of the electrolysis hydrogen as at least part of the hydrogen used in the synthesis unit; and
supplying at least a portion of the electrolysis oxygen as at least part of the oxygen used in the partial oxidation unit.

2. The method of claim 1, wherein the biomass comprises manure, organic waste, sewerage and/or cellulose.

3. The method of claim 1, wherein the anaerobic digester further produces sulphur and/or fertilizer.

4. The method of claim 1, further comprising heating the anaerobic digester using by-product heat generated by the partial oxidation unit.

5. The method of claim 1, further comprising heating the anaerobic digester using by-product heat generated by the water electrolysis unit.

6. The method of claim 1, wherein the oxygen supplied to the partial oxidation unit consists of the electrolysis oxygen.

7. (canceled)

8. The method of claim 1, wherein the syngas supplied to the synthesis unit consists of the syngas produced by the partial oxidation unit.

9. The method of claim 1, wherein the hydrogen supplied to the synthesis unit consists of the electrolysis hydrogen.

10. (canceled)

11. The method of claim 1, wherein the water electrolysis unit further produces deuterium.

12. The method of claim 11, wherein at least a portion of the deuterium is supplied to a nuclear reactor facility.

13. The method of claim 1, wherein:
during electricity demand over an upper value:
powering the water electrolysis unit using electricity obtained from a generator fuelled with a portion of the stored bio-methanol;
during electricity demand below a lower value:
powering the water electrolysis unit, and optionally hydrogen and oxygen compressors, using electricity obtained from a source supplied by renewable and/or nuclear energy sources and/or from independent renewable electricity generators.

14. The method of claim 1, wherein the base threshold is relatively constant and pre-determined.

15. The method of claim 13, wherein the upper and lower values are the same.

16. The method of claim 15, wherein the upper and lower values and the base threshold are the same.

17. The method of claim 1, further comprising regulating the base threshold over time to maintain overall greenhouse gas neutrality of the process.

18. The method of claim 1, further comprising:
controlling electricity input into the water electrolysis unit and controlling the electricity generation from the bio-methanol to maintain overall greenhouse gas neutrality of the process, and reducing negative impacts of electricity demand characteristics.

19. A system for producing bio-methanol, comprising:
an anaerobic digester unit for producing biogas comprising methane and carbon dioxide;
a partial oxidation unit for receiving the biogas and configured to produce syngas;
a synthesis unit for receiving the syngas and hydrogen, and configured to produce bio-methanol;
a power control assembly configured to
supply at least a portion of the bio-methanol to a generator for producing electricity, during critical electricity demand over an upper threshold; and
supply at least a portion of the bio-methanol to storage for use as transportation fuel or as a commercial or industrial alcohol, during electricity demand below a lower threshold;
a water electrolysis unit to produce oxygen and hydrogen;
a hydrogen supply and storage assembly configured to supply at least a portion of the electrolysis hydrogen as at least part of the hydrogen used in the synthesis unit; and
an oxygen supply and storage assembly configured to supply at least a portion of the electrolysis oxygen as at least part of the oxygen used in the partial oxidation unit.

20.-21. (canceled)

22. A process for integrating a water electrolysis unit and bio-methanol storage facility:
monitoring electricity demand;
during electricity peak demand:
diverting bio-methanol from storage to electricity generation to produce methanol-generated electricity;
reducing or ceasing system electricity utilization for operating the water electrolysis unit; and
utilizing the methanol- and/or biogas-generated electricity for operating the water electrolysis unit; and
during electrical system demand below the peak:
storing bio-methanol produced by a bio-methanol production facility for distribution;
ceasing generation of the methanol-generated electricity; and
increasing use of the system electricity for the water electrolysis unit.

23. (canceled)
